(19) United States
(12) Patent Application Publication
(10) Pub. No.: US 2025/0110131 A1
(43) Pub. Date: Apr. 3, 2025

(54) BREAST IMPLANT-ASSOCIATED ANAPLASTIC LARGE CELL LYMPHOMA DIAGNOSTIC TEST

(71) Applicants: THE TRUSTEES OF INDIANA UNIVERSITY, Bloomington, IN (US); RHODE ISLAND HOSPITAL, Providence, RI (US)

(72) Inventors: Mithun SINHA, Indianapolis, IN (US); Imran Khan MOHAMMED, Indianapolis, IN (US); Marshall E. KADIN, Stuart, FL (US)

(21) Appl. No.: 18/832,947

(22) PCT Filed: Feb. 1, 2023
(86) PCT No.: PCT/US2023/012086

§ 371 Date: Jul. 25, 2024

Related U.S. Application Data
(60) Provisional application No. 63/305,759, filed on Feb. 2, 2022.

(51) Int. Cl. G01N 33/574; G01N 33/543; G01N 33/68

(52) U.S. Cl. CPC G01N 33/57415; G01N 33/54388; G01N 33/57484; G01N 33/6869; G01N 33/6872; G01N 2333/5425; G01N 2333/5428; G01N 2333/5437; G01N 2333/70578

(57) ABSTRACT

Breast implant-associated anaplastic large cell lymphoma (BIA-ALCL) is a rare T-cell lymphoma that can develop around breast implants. The disclosure is directed towards devices and methods for diagnose BIA-ALCL from the seroma (fluid) surrounding the implant using a lateral flow assay (LFA) detecting CD30 and/or one or more cytokines known to be produced by tumor cells in BIA-ALCL.

### CROSS REFERENCE TO RELATED APPLICATIONS

This application claims priority to U.S. Provisional Patent Application No. 63/305,759, filed on Feb. 2, 2022, the disclosure of which is expressly incorporated herein.

### BACKGROUND

Breast implant-associated anaplastic large cell lymphoma (BIA-ALCL) is a rare T-cell lymphoma that can develop around breast implants. BIA-ALCL can present as a solid mass infiltrating the peri-prosthetic fibrotic capsule and soft tissues, but more commonly as a late peri-implant effusion (seroma). Affected individuals rarely present with symptoms of skin rash, fever night sweats and lymphadenopathy. Diagnosis is based on ultrasound-guided, fine-needle aspiration of the peri-implant fluid, together with immunohistochemistry for CD30+ large anaplastic T-cells. The disease has been associated with both silicone and saline implants374861 in aesthetic (breast augmentation) as well as reconstructive (post-mastectomy) patients. Without access to breast implants, many cancer survivors would not feel they have returned to a full, complete life. The delay between implant insertion and diagnosis of BIA-ALCL has ranged from 0.8 years to about 27 years, with a mean of about 9.75 years. BIA-ALCL was recognized by the World Health Organization in 2016 and was provisionally defined as a non-Hodgkin's lymphoma. Per US FDA, as of April 2022, 1,130 cases of BIA-ALCL have been reported with 59 deaths. However, the numbers are believed to be grossly under reported. There are 10 million women with breast implant worldwide who are at risk of developing this malignant lymphoma. FDA is providing regular updates on adverse events due to the breast implant including BIA-ALCL. The current challenge lies in understanding the cause and early treatment of the malignant lymphoma. Thus, it is extremely important to understand the mechanism at the cellular level towards development of diagnostics and therapy regimen for this relatively new type of lymphoma.

The role of cytokines in the pathogenesis and metastasis of this lymphoma is receiving attention. Importantly patient survival is significantly increased with early detection and en bloc resection of the implant and surrounding fluid and capsule. However, currently, there are no diagnostic tools for early detection of BIA-ALCL. This disclosure is directed towards systems and methods to diagnose BIA-ALCL from the seroma surrounding the implant using lateral flow assays.

### SUMMARY

In the present disclosure, several cytokines have been identified to be associated with BIA-ALCL. These cytokines are detected at significantly higher levels in 8 malignant seromas compared to 40 benign seromas. Detection of IL-10, IL-9, IL, 13, and/or CD30, can help in early diagnosis of lymphoma, prior to metastasis of tumor cells, and may be used to initiate treatment (e.g., curative treatment) for patients.

This disclosure is directed towards systems and methods to diagnose BIA-ALCL from the seroma surrounding the implant using a lateral flow assay (LFA) as described. An assay or test has been developed for measuring IL-10, IL-9, IL-13, CD30, and similar associated antigens as point of care (POC) diagnostics for BIA-ALCL. The developed assay or test can be easily performed by care provider (surgeon, physician, nurse etc.) during or after surgery. The results can be obtained in less than about 20 minutes.

As disclosed herein, expression of IL-10, IL-9, IL-13, CD30, and other associated cytokines in the seroma of BIA-ALCL patients may be diagnostic markers for BIA-ALCL and/or may be used to identify benign cellular precursor(s) of BIA-ALCL.

In accordance with one embodiment, a method of treating subjects at high risk for developing breast implant-associated anaplastic large cell lymphoma (BIA-ALCL) is described. The method comprises obtaining a peri-implant fluid sample from a subject, identifying the subject at risk of BIA-ALCL by measuring an antigen in the fluid from the subject by use of a point-of-care test for a prescribed time period. The subject will be determined to have BIA-ALCL when the concentration of the antigen level causes a positive indication in the point-of-care test. If a positive indication is recorded, staging studies (e.g. radiographs including PET scan) may be performed.

In one embodiment, the time period ranges from about 1 minute to about 20 minutes. In one embodiment, the biomarker is used to identify a benign cellular precursor of BIA-ALCL. In one embodiment, the body fluid is obtained from a seroma of the patient. In one embodiment, the antigen is selected from IL-9, IL-10, IL-13, and CD30. In one embodiment, the antigen comprises a first antigen and a second antigen, wherein the first antigen and the second antigen are selected from IL-9, IL-10, IL-13, and CD30. In one embodiment, the antigen comprises a first antigen, a second antigen, and a third antigen, wherein the first antigen, the second antigen, and the third antigen are selected from IL-9, IL-10, IL-13, and CD30.

In one embodiment, the method of determining that the subject has BIA-ALCL comprises using a lateral flow assay. In one embodiment, the method of determining that the subject has BIA-ALCL comprises using a multiplex lateral flow assay.

In accordance with one embodiment of the present disclosure, a method of treating breast implant-associated anaplastic large cell lymphoma (BIA-ALCL) in a patient is described. The method comprises analyzing the seroma sample, using a multiplex lateral flow assay, for the presence of one or more antigens, diagnosing the patient as having BIA-ALCL based on the detected levels of the one or more antigens in the sample, and administering a therapeutic if the subject has BIA-ALCL.

In one embodiment, the antigen comprises a first antigen, a second antigen, and a third antigen, wherein the first antigen, the second antigen, and the third antigen are selected from IL-9, IL-10, IL-13, and CD30. In one embodiment, the multiplex lateral flow assay comprises dots of each capture antibody for the first antigen, the second antigen, and the third antigen. In one embodiment, the multiplex lateral flow assay comprises a radial arrangement of multiple single antigen based lateral flow assays.

In accordance with one embodiment of the present disclosure, a device for diagnosing breast implant-associated anaplastic large cell lymphoma (BIA-ALCL) in a patient is described. The device comprises a multiplex lateral flow assay comprising capture antibodies for the one or more antigens used as markers for BIA-ALCL.

In one embodiment, the devices comprises a first antigen, a second antigen, and a third antigen, wherein the first antigen, the second antigen, and the third antigen are selected from IL-9, IL-10, IL-13, and CD30. In one embodiment, the multiplex lateral flow assay comprises dots of each capture antibody for the first antigen, the second antigen, and the third antigen. In one embodiment, the multiplex lateral flow assay is designed to measure IL-10 and IL-13, and wherein if IL-10 is measured to be greater than about 150 pg/ml and IL-13 is measured to be greater than about 714 pg/ml, the device diagnoses BIA-ALCL in the patient.

In one embodiment, the device comprises a first antigen and a second antigen wherein, the first antigen is selected from IL-9, IL-10, and IL-13, and the second antigen is CD30. In one embodiment, the device comprises a first antigen that can identify a benign cellular precursor of for BIA-ALCL. In one embodiment, the multiplex lateral flow assay is comprised of a kit comprising reagents for measuring the antigen(s).

### BRIEF DESCRIPTION OF THE DRAWINGS

These and other features, aspects, and advantages of the present disclosure will become better understood when the following detailed description is read with reference to the accompanying drawings, in which like characters represent like parts throughout the drawings.

FIG. 1 is a graphical illustration showing increased IL-9 in the seroma of BIA-ALCL subjects compared to that of benign subjects. BIA-ALCL cell line TBLR1 supplements were used as positive controls.

FIG. 2A is a schematic of a lateral flow assay (LFA) before application of the analyte.

FIG. 2B is a schematic of a lateral flow assay (LFA) post-application of analyte.

FIG. 3A is an illustration of successful optimization of IL-9 LFA using the kit to detect with 1 ng/ml recombinant human IL-9 with monoclonal antibodies.

FIG. 3B is an illustration of successful optimization of IL-9 LFA using the kit to detect 20 ng/ml human recombinant with IL-9 antibodies.

FIG. 3C is an illustration of successful optimization of IL-9 LFA using the kit to detect IL-9 antigen in culture supernatants from BIA-ALCL derived cell line TLBR1 with anti-IL9 monoclonal antibodies.

FIG. 4A is an illustration of successful optimization of CD30 LFA using the kit with 1 ng/ml recombinant human CD30 antigen with anti-CD30 monoclonal antibodies.

FIG. 4B is an illustration of successful optimization of CD30 LFA using the kit to detect 2 ng/ml recombinant human CD30 antigen with monoclonal CD30 antibodies.

FIG. 5 includes images of the CD30 LFA assay used to distinguish BIA-ALCL from benign seromas.

FIG. 6A is a bar graph depicting an increased IL-10 in a sample. The graph also illustrates its ROC to determine specificity and sensitivity.

FIG. 6B is a bar graph depicting an increased IL-13 in a sample. The graph also illustrates its ROC to determine specificity and sensitivity.

FIG. 6C includes images of the IL-10 LFA assay used to distinguish BIA-ALCL from benign seromas.

### DETAILED DESCRIPTION

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “subject” means an animal including but not limited to humans, domesticated animals including horses, dogs, cats, cattle, and the like, rodents, reptiles, and amphibians.

As used herein, the term “patient” without further designation is intended to encompass any warm blooded vertebrate domesticated animal (including for example, but not limited to livestock, horses, cats, dogs, and other pets) and humans receiving a therapeutic treatment whether or not under the supervision of a physician.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein, the term “biomarker,” “marker,” “antigen,” or “molecular marker,” is a biological molecule found in blood, urine, other body fluids such as lymph fluid or breast milk, seroma, or tissues that is a sign of a normal or abnormal process, or of a condition or disease. A biomarker may be a protein, a peptide, a gene, a cytokine, a metabolite, a cell, or any other biologically relevant material. A biomarker may be used to see how well the body responds to a treatment for a disease or condition. A biomarker may be used to predict a disease, predict an early onset of disease, or to predict relevant clinical outcomes across a variety of treatments and populations. These substances can be found in the blood, urine, stool, tumor tissue, serum, or other tissues or bodily fluids of patients. In particular here, the biomarkers are found in fluid around the breast implant.

In one embodiment, the biomarker may indicate a disease state in the patient. In one embodiment, the disease is cancer. In one embodiment, the cancer is BIA-ALCL. In one embodiment, the disease is early stage BIA-ALCL.

In one embodiment, a method can be employed to identify and measure an antigen indicating BIA-ALCL. In one embodiment, a method can be employed to identify and measure an antigen indicating BIA-ALCL in the seroma of the patient. In one embodiment, the method further comprises monitoring the patient for BIA-ALCL. In one embodiment, the method further comprises determining if the patient is eligible for a therapeutic for BIA-ALCL.

In one embodiment, administering the therapeutic comprises administering the patient with a drug. In one embodiment, administering the preventative therapeutic comprises administering the patient with a therapeutic regimen that affects patient behavior including but not limited to altering diet or fluid intake. In one embodiment, the therapeutic comprises en bloc resection of the implant, capsule, and seroma fluid.

Multiple cytokines have been identified in the analysis of seromas used to diagnose BIA-ALCL. Lymphoma cell culture supernatants and seromas from 8 patients with BIA-ALCL were compared with 9 benign seromas using the LEGENDplex Human Thelper (Th) Cytokine Panel (13-plex). The analysis showed a clear distinction in that only malignant seromas resembled culture supernatants of BIA-ALCL cell lines, particularly with respect to higher concentrations of IL-10, IL-13, and IL-9. Concentrations of IFNγ greater than about 1000 pg/ml also distinguished BIA-ALCL from benign seromas. IL-6, known to be a driver of malignant cells, was elevated in benign seromas and did not distinguish them from malignant seromas. Cytokine concentration was shown to be proportional to tumor cell number in cell culture supernatants and tumor cell production of IL-6, IL-9, IL-10, IL-13 were confirmed by flow cytometry and immunocytochemistry. Additionally, receiver operating characteristic (ROC) curves showing a cutoff of about 39.03 pg/mL for IL-10 and of about 68.05 pg/mL for IL-13 were both associated with sensitivity (Se) of about 100% and specificity (Sp) of about 100% (Youden index=1), for diagnosis of BIA-ALCL. IL-9 has been reported to induce proliferation and metastasis in hepatocellular carcinoma by activating the JAK2/STAT3 pathway. BIA-ALCL has also been associated with increased IL-9, IL-10, and/or IL-13 in seromas surrounding implants. Recently, in a study of 25 patients with BIA-ALCL and 30 patients with benign seromas, analysis illustrated a cutoff of 150 pg/mL for IL-10 with a sensitivity of 0.92 and specificity of 1 for a Youden index of 0.92; and for IL-13 a cutoff of 714 pg/ml with sensitivity of 0.76, and specificity of 0.97 for a Youden index of 0.73.

In accordance with one embodiment, expression of IL-9, CD30 and/or other associated cytokines in the seroma of BIA-ALCL patients may serve as a diagnostic marker for BIA-ALCL. In accordance with one embodiment, expression of IL-9, CD30 and/or other associated cytokines in the seroma of BIA-ALCL patients may be utilized to identify a benign cellular precursor or BIA-ALCL. In accordance with one embodiment, expression of IL-9, CD30 and/or other associated cytokines in the seroma of BIA-ALCL patients may be utilized for early diagnosis of BIA-ALCL prior to metastasis of tumor cells.

In accordance with one embodiment, systems and methods are directed towards developing assays for IL-9, CD30 and/or similar associated antigens or biomarkers as point of care (POC) diagnostics for BIA-ALCL detection. In some embodiments, the assay may be a Lateral Flow Assay (LFA), also known as a dipstick test. A LFA is used to detect chorionic gonadotropin for pregnancy testing and for identifying visceral leishmaniasis by measuring IL-6. Thus, no special skill set is required in the employment of a LFA. A LFA can be easily performed by care provider (surgeon, physician, or nurse) during or after surgery. The results can be obtained in about twenty minutes or less. In some embodiments, results from a LFA may be obtained in less than about 1 minute.

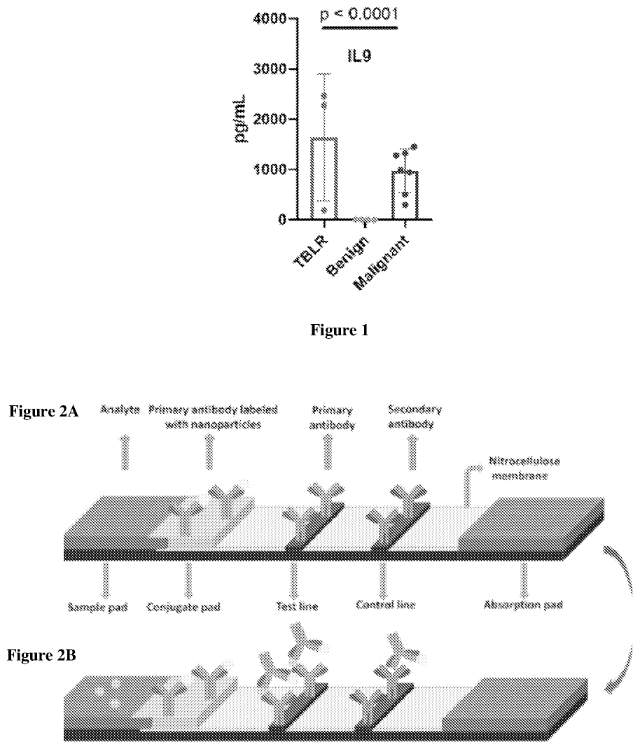

In accordance with one embodiment, as shown in FIG. 1, a significant difference in IL-9 levels is observed for malignant and benign seromas. Supernatants of BIA-ALCL cell lines (TLBR) were used as positive controls. The results illustrated in FIG. 1 were obtained by flow cytometric analysis of beads binding a panel of 13 cytokines. However, such tests are expensive and can be done only in a reference laboratory with special equipment and experienced personnel.

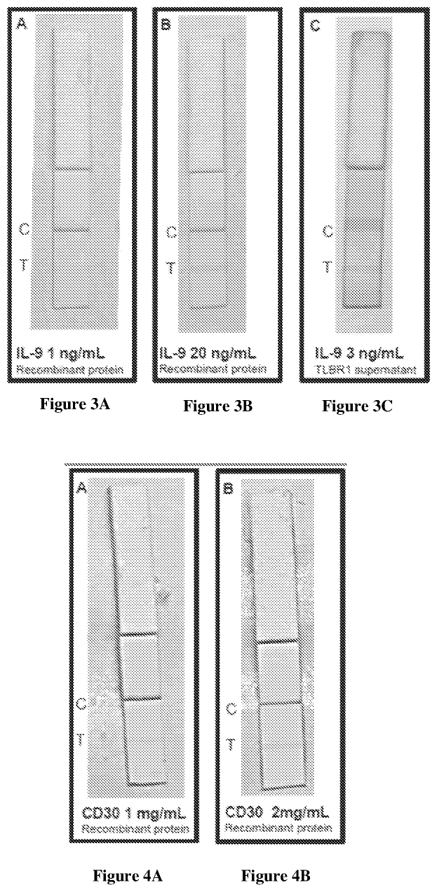

In accordance with one embodiment, a model lateral flow assay for IL-9 and CD30 with the long-term goal of establishing point of care detection of BIA-ALCL was established. In accordance with one embodiment, as shown in FIGS. 3A-3B, about 20 ng of recombinant IL-9 and about 1 ng of recombinant IL-9 protein were detected. In accordance with one embodiment, as shown in FIG. 3C, about 3 ng/ml of IL-9 in cell culture supernatant of BIA-ALCL line TLBR1 was detected.

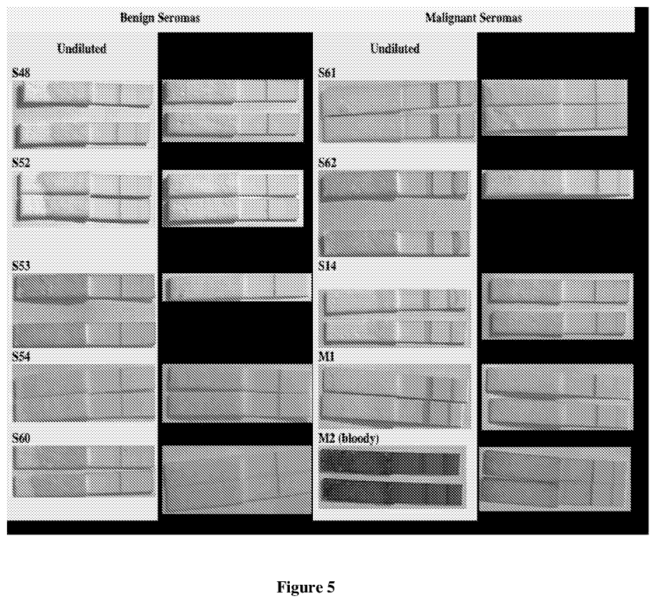

In accordance with one embodiment, as shown in FIGS. 4A-4B, about 1 mg and about 2 mg recombinant CD30 protein were detected. In accordance with one embodiment, as shown in FIG. 5, CD30 in the seroma of a BIA-ALCL patient was detected.

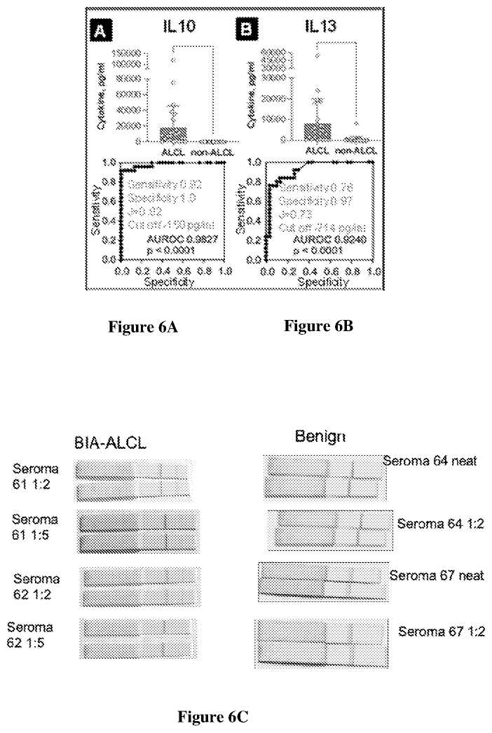

In accordance with one embodiment, as shown in FIG. 5, CD30 LFA was used to distinguish five BIA-ALCL seromas from five benign seromas. In accordance with one embodiment, as shown in FIG. 6C, IL-10 LFA was used to distinguish BIA-ALCL seromas from benign seromas. A positive test line was found in undiluted BIA-ALCL seromas and in BIA-ALCL seromas with 1:10 dilutions. However, a positive test line was not detected any benign seroma with 1:10 dilution. Three of five benign seromas had a faint positive test line when undiluted. Thus, in accordance with one embodiment, seroma from patients may be diluted prior to CD30 LFA.

In accordance with one embodiment, LFAs are developed for detection of a single analyte per assay. In accordance with one embodiment, LFAs are developed for detection of multiple analytes per assay. For example, simultaneous measurement of multiple analytes from a single sample may be conducted for achieving efficient and high-throughput detection of cancer. Multiplexed immunobead-based cytokine profiling of patient sera may be used for early detection of ovarian cancer. Abdominal fluid (ascites) may be present in more than one-third of patients with ovarian cancer. In some embodiments, simultaneous measurement of multiple analytes is critical because a false negative/positive result based on a single target could have serious consequences. However, by having multiple targets, the probability of false negative/positive results are reduced when multiple analytes are analyzed per assay. Furthermore, multiplexing has added benefits including improved efficiency of testing and reduced costs.

In accordance with one embodiment, a multiplexed LFA with each of two cytokines in combination with CD30 may be designed. Preliminary data from BIA-ALCL (N=25) vs benign seromas (N=30) shows that mean concentrations of IL-10, IL-9 and IL-13 were significantly higher (p<0.0001) in BIA-ALCL seromas compared with benign seromas. IFNγ was also significantly higher in BIA-ALCL (p=0.01). In accordance with one embodiment, a minimal amount of IL-10 and IL-13 can be used to separate BIA-ALCL from benign seromas was established to provide a guide for the multiplex LFA. In some embodiments, if IL-10 was determined to be greater than about 150 pg/ml (20/22 subjects with BIA-ALCL (91%)) and IL-13 was determined to be greater than about 714 pg/ml (19/25 subjects (76%)), the measurements may be diagnostic of BIA-ALCL. Thus, in some embodiments, a combination of cytokines increased the sensitivity of BIA-ALCL detection. As shown in FIGS. 6C, IL-10 is not detected in benign seroma #64, #67 but IL-10 is detected in malignant seroma #61, #62, which was also positive for CD30.

In accordance with one embodiment, a system for detecting early stage BIA-ALCL comprises a multiplexed LFA designed to measure two antigens selected from a group of IL-10, IL-9, IL-13, and CD30. In accordance with one embodiment, a system for detecting early stage BIA-ALCL comprises a multiplexed LFA designed to measure a first antigen selected from a group of IL-10, IL-9, and IL-13, and a second antigen comprising CD30.

In accordance with one embodiment, the present disclosure is directed to a kit comprising a bottle comprising reagents and antibodies associated with diagnosing or detecting BIA-ALCL. The kit can further include LFA paper strips. The kit can further include comprising a diluent. The kit can further include instructions for use. The kit can include materials and reagents for a single use or for use with multiple patient samples.

### Example 1: Lateral Flow Assay (LFA)

The development of Lateral Flow Assay or Lateral Flow Immunochromatography Assay can be divided into two steps: (1) standardizing membrane characteristics, and (2) optimizing molecular level immunoassay reaction between analyte and detector molecules. In the preliminary phase the reaction specificity of capture and detector antibodies with the analyte may be confirmed with other techniques like ELISA. Molarity and pH of conjugation buffer are also be an important considerations for the immunoreaction between the analyte and antibodies. Epitope mapping of the capture and detector antibodies may also be required to confirm the specificity of the assay. Standardization of membrane characteristics directly relates to the sensitivity of the assay based on its porosity, hydrophobicity, protein holding/releasing capacity and wicking rate. Under optimized conditions, a perfect Lateral Flow Immunochromatography Assay may have high on-rate (target binding efficiency), low off-rate (target releasing efficiency) and low cross reactivity. The key component of this assay includes a nitrocellulose membrane, a detector reagent, and gold nanoparticles.

Nitrocellulose membranes may be considered to be the backbone of any rapid test strip coated with capture antibodies for a test line and a control line. The size of the analyte and sample type (whole blood, serum, plasma, or urine etc.) may be considered while determining the pore size of the nitrocellulose membrane. As the pore size increase the flow rate of analyte through the membrane also increases. Sensitivity of the assay and pore size are inversely proportional. The pH of the buffer for capture reagents may be optimized based on the isoelectric point of the protein used in the assay in order to enhance the electrostatic interaction of the protein with the nitrocellulose membrane.

In some embodiments, the pore size of nitrocellulose membrane may range from about 1 micrometer to about 20 micrometers, including any size and range of size comprised therein. For example, the pore size of nitrocellulose membrane may range from about 1 micrometer to about 5 micrometer, about 5 micrometer to about 10 micrometer, or about 15 micrometer to about 20 micrometer. In some embodiments, the pore size of nitrocellulose membrane may be selected based on the analyte characteristics. Nitrocellulose membranes may be rated either by pore size or by a wicking time. As the pore size decreases the wicking time increases, which offers adequate time for antigen-antibody interaction, and thereby enhances sensitivity of the assay.

The detector reagent may be responsible for the color formation in LFA due to aggregation of nanoparticles taking place during assay reaction. Nanoparticles (e.g., gold nanoparticles) may be used for conjugation with antibodies because of their inert nature and/or because they comprise a perfect spherical structure. In some embodiments, the size of the gold nanoparticles may range from about 20 nm to about 40 nm, about 40 nm to about 60 nm, about 60 nm to about 80 nm, about 80 nm to about 100 nm, about 100 nm to about 120 nm, about 120 nm to about 140 nm, about 140 nm to about 160 nm, about 160 nm to about 180 nm, or about 180 nm to about 200 nm including any size or range of size comprised therein. For example, gold nanoparticles used for the conjugation purpose may comprise a size of about 40 nm. The change in particle size of the gold nanoparticles may be analyzed by an absorbance scan in spectrophotometer. Color of the gold nanoparticles may change with a change in size.

Gold nanoparticles are used for antibody conjugation. Conjugation efficiency of the detector antibody to the gold nanoparticles may be considered as a rate limiting process in the successful development of LFA. The pH of the conjugation buffer may be optimized such that the antibody molecules completely bind to the colloidal gold nanoparticles.

### Example 2: Assay Development

An LFA kit (abcam #ab270537) was used with (A) IL-9 monoclonal antibody pair that permits detection of separate epitopes of human IL-9 (abcam #ab256613) with recombinant human IL-9 as test analyte (ab #214417), and (B) CD30 monoclonal antibody pair that permits detection of separate epitopes of human CD30 (abcam #ab244142) with recombinant human CD30 as test analyte (abcam #ab140584). The abcam LFA kit is antibody agnostic and may be a preferred platform for optimizing and utilizing new LFA assays.

The abcam LFA kit comprises a LFA paper strip that includes a sample pad, conjugation pad, detection pad, and absorbance pad (see, FIG. 2A-2B). The sample pad ensures controlled flow of the test solution (e.g., seroma fluid), which migrates to the conjugate pad where nanoparticles labeled with antibodies are stored. If the target analyte (e.g., IL-9 or CD30) is present, the labeled antibodies will bind to them and continue to migrate to the detection pad. The target analyte is captured by immobilized antibodies at a test line (T-line) to form a colored stripe while a subsequent control line (C-line) is used to indicate that the solution has sufficiently migrated. The colorimetric detection is obtained through gold nanoparticles conjugated to IL-9 or CD30 antibodies to give a red color. Finally, the absorbent pad absorbs any excess sample. The IL-9 or CD30 antibody pair is comprised of capture and detection antibodies. The capture antibodies were conjugated to an Ulfa-Tag provided in the ab270537 kit. The lysine present is antibody is targeted and cross-linked to the Ulfa-Tag. IL-9 and/or CD30 antibodies are simultaneously detected by conjugation with the gold nanoparticles.

### Example 3: Multiplexing Assay Development

The process of multiplexing may be achieved in two steps. First, individual LFAs will be developed for two/three cytokines (e.g., IL-10, IL-13 and IL-9). The individual LFA for each cytokine will be developed using strategies as described below. The antibody pair for each cytokine will be optimized separately with capture antibody conjugated to ULFA-tag and detection antibody conjugated to 40 nm gold nanoparticles. Anti-cytokine antibodies must bind to different cytokine epitopes. The LFA will be optimized on MDI70 nitrocellulose membrane. An LFA will be first developed for IL-10 to ensure that high concentration of IL-10 in 30 benign seromas (about 47 pg/ml) will not overlap with the mean concentration of IL-10 in 25 malignant seromas (about 17900 pg/ml). Next, an LFA for IL-13 will be developed. IL-13 was detected with a high concentration of about 1856 pg/ml in benign seromas compared with to a mean concentration of about 8096 in malignant seromas. Alternatively, or additionally, an LFA may be used to measure IL-9 concentrations.

Second, after successfully optimizing LFAs for the above identified cytokines, the LFAs will be used with CD30 in a multiplex LFA. Development of multiplex LFAs can be achieved either through spatial separation of lines in one strip by introducing dots of each capture antibody or by a radial arrangement of multiple single antigen based LFAs.

In a preferred embodiment, the multiplex LFA will be developed with the first approach, whereby a single strip with six dots corresponding to duplicates of CD30, IL-10, and IL-13 will be used. Dot multiplexing is preferred over radial multiplexing because it requires a limited amount of implant seroma which is typically collected in small quantities (about 100 μl). Radial multiplex may be more suitable for analytes collected in higher volumes (e.g., from urine). Additionally, dot multiplexed LFA are less expensive and more convenient to handle.

### Example 4: Generating multiplex LFA by Combining Cytokines With CD30

Multiplexing will be initiated using IL-10 as the cytokine of choice because of its high sensitivity and specificity (Youden Index 0.92) for BIA-ALCL. A positive test line may be obtained with recombinant IL-10 protein concentration at a cut-off value of about 150 pg/ml. Different concentrations of detector antibodies may be used. Additionally, or alternatively, larger gold nanoparticles or carbon nanoparticles may be used increase sensitivity. Subsequently, the assay may be used with recombinant IL-13 protein concentration at a cutoff level of about 714 pg/ml. Once the cytokine with high sensitivity in LFA is identified, a multiplex LFA may be designed in combination with CD30.

1. A method of treating subjects at risk for developing breast implant-associated anaplastic large cell lymphoma (BIA-ALCL), said method comprising:
obtaining a body fluid sample from a subject;
identifying the subject at risk of BIA-ALCL by measuring an antigen in a body fluid of the subject by using a point-of-care test for a prescribed time period;
determining that the subject has BIA-ALCL when the concentration of the antigen level causes a positive indication in point-of-care test; and
administering a BIA-ALCL therapeutic if the subject has BIA-ALCL.

2. The method of claim 1, wherein the time period ranges from about 1 minute to about 20 minutes.

3. The method of claim 1, wherein the biomarker in used to identify a benign cellular precursor of BIA-ALCL.

4. The method of claim 1, wherein the body fluid is obtained from a seroma of the patient.

5. The method of claim 1, wherein the antigen is selected from IL-10, IL-13, IL-9, and CD30.

6. The method of claim 1, wherein the antigen comprises a first antigen and a second antigen, wherein the first antigen and the second antigen are selected from IL-10, IL-13, IL-9, and CD30.

7. The method of claim 1, wherein the antigen comprises a first antigen, a second antigen, and a third antigen, wherein the first antigen, the second antigen, and the third antigen are selected from IL-10, IL-13, IL-9 and CD30.

8. The method of claim 1, wherein the method of determining that the subject has BIA-ALCL comprises using a lateral flow assay.

9. The method of claim 8, wherein the method of determining that the subject has BIA-ALCL comprises using a multiplex lateral flow assay.

10. A method of treating breast implant-associated anaplastic large cell lymphoma (BIA-ALCL) in a patient said method comprising:
obtaining a seroma sample from fluid surrounding an implant in the patient;
analyzing the seroma sample, using a multiplex lateral flow assay, for the presence of an antigen;
diagnosing the patient as having BIA-ALCL based on the detected levels of the antigen in the sample; and
administering a therapeutic if the subject has BIA-ALCL.

11. The method of claim 10, wherein the antigen comprises a first antigen, a second antigen, and a third antigen, wherein the first antigen, the second antigen, and the third antigen are selected from IL-10, IL-13, IL-9, and CD30.

12. The method of claim 11, wherein the multiplex lateral flow assay comprises dots of each capture antibody for the first antigen, the second antigen, and the third antigen.

13. The method of claim 11, wherein the multiplex lateral flow assay comprises a radial arrangement of multiple single antigen based lateral flow assays.

14. A device for diagnosing breast implant-associated anaplastic large cell lymphoma (BIA-ALCL) in a patient comprising:
a multiplex lateral flow assay comprising capture antibodies for an antigen used as marker for BIA-ALCL.

15. The device of claim 14, comprising a first antigen, a second antigen, and a third antigen, wherein the first antigen, the second antigen, and the third antigen are selected from IL-10, IL-13, IL-9, and CD30.

16. The device of claim 15, wherein the multiplex lateral flow assay comprises dots of each capture antibody for the first antigen, the second antigen, and the third antigen.

17. The device of claim 14, wherein the multiplex lateral flow assay is designed to measure IL-10 and IL-13, and wherein if IL-10 is measured to be greater than about 150 pg/ml and IL-13 is measured to be greater than about 714 pg/ml, the device diagnoses BIA-ALCL in the patient.

18. The device of claim 14, comprising a first antigen and a second antigen wherein, the first antigen is selected from IL-10, IL-13, and IL-9 and the second antigen is CD30.

19. The device of claim 14, comprising a first antigen that is can identify a benign cellular precursor of for BIA-ALCL.

20. The device of claim 14, wherein the multiplex lateral flow assay is comprised in a kit comprising reagents for measuring the antigen.
